(19) United States
(12) Patent Application Publication
(10) Pub. No.: US 2023/0371813 A1
(43) Pub. Date: Nov. 23, 2023

(54) THREE-DIMENSIONAL OCULAR ENDOSCOPE DEVICE AND METHODS OF USE

(71) Applicant: EyeQ Technologies, Inc., Reno, NV (US)

(72) Inventors: Matthew Schuster, Reno, NV (US); Brendan Reese, Reno, NV (US); Luke W. Clauson, Reno, NV (US)

(21) Appl. No.: 18/319,316

(22) Filed: May 17, 2023

Related U.S. Application Data
(60) Provisional application No. 63/342,631, filed on May 17, 2022.
(60) Provisional application No. 63/357,963, filed on Jul. 1, 2022.

(51) Int. Cl. A61B 3/14; A61B 1/07; A61B 1/06; A61B 1/04

(52) U.S. Cl. CPC A61B 3/14; A61B 1/07; A61B 1/0669; A61B 1/042; A61B 1/313

(57) ABSTRACT

An endoscopic device for providing three-dimensional visualization of an intraocular object and having a first portion coupled to a second portion. The first portion includes a distal shaft having an outer diameter sized for positioning in an intraocular space, an illumination guide extending through the distal shaft, at least one image transmitter extending through the distal shaft, and at least one objective lens positioned within the distal shaft at a distal end of the at least one image transmitter. The second portion includes a housing, an illumination source positioned within the housing and in optical communication with a proximal end of the illumination guide, and at least one image sensor positioned within the housing and in optical communication with the proximal end of the at least one image transmitter. Related devices, systems, and methods are provided.

### CROSS-REFERENCE TO RELATED APPLICATIONS

This application claims the benefit of priority under 35 U.S.C. § 119(e) to co-pending U.S. Provisional Patent Application Ser. No. 63/342,631, filed May 17, 2022 and co-pending U.S. Provisional Patent Application Ser. No. 63/357,963, filed Jul. 1, 2022. The disclosures of the applications are incorporated by reference in their entirety.

### BACKGROUND

Endoscopes are visualization tools that allow imaging of the body's internal structures. Endoscopes can be used in procedures across many disciplines of medicine, such as otology, pulmonology, otolaryngology, and intravascular neurosurgery, among other areas, for various procedures, such as such as ophthalmic endoscopy, otoscopy, cystoscopy, nephroscopy, bronchoscopy, arthroscopy, colonoscopy, and laparoscopy. Providing visualization at the site of a surgery can reduce complications due to surgeon error in locations that have historically been difficult to visualize. In certain areas, such as inside the eye, significant size limitations exist. Conventional endoscopes for ocular surgeries must have small diameters and are typically no larger than an 18-gauge hypodermic needle (0.050 inches in diameter), which is much smaller than endoscopes used in areas of the body without such size limitations. Commercially available ophthalmic endoscopic systems currently provide two dimensional images due to their small size, yet there would be significant advantage in being able to visualize the anatomy of the eye three-dimensionally.

In view of the foregoing, there is a need for improved devices and methods related to endoscopes for ophthalmic surgery.

### SUMMARY

In an aspect, described is an endoscopic device configured to provide three-dimensional visualization of an intraocular object. The device includes a first portion including a distal shaft having a lumen extending along a longitudinal axis, an outer diameter sized for positioning in an intraocular space, an illumination guide extending through the lumen of the distal shaft, at least one image transmitter extending through the lumen of the distal shaft, and at least one objective lens positioned within the lumen at a distal end of the at least one image transmitter. The device includes a second portion coupled to the first portion and the second portion includes a housing, an illumination source positioned within the housing in optical communication with a proximal end of the illumination guide, and at least one image sensor positioned within the housing in optical communication with a proximal end of the at least one image transmitter.

The at least one objective lens can be a single objective lens having an optical axis, The single objective lens can be configured to spatially translate relative to the distal shaft between at least a first location and at least a second location horizontally off-set from the first location. The device can be configured to capture images of the intraocular object as the single objective lens spatially translates to provide the three dimensional visualization. A distance between an optical axis of the single objective lens when positioned at the first location to the optical axis of the single objective lens when positioned at the second location can be proportional to a working distance of the endoscope such that the distance can define a parallax angle for a distance from the first and second locations to the intraocular object. A subtended parallax angle can form two images of the intraocular object at different angular perspectives. The spatial translation of the single objective lens can occur side-to-side from the at least a first location to the at least a second location. The spatial translation of the single objective lens can be rotational around the longitudinal axis from the at least a first location to the at least a second location, and the single objective lens can be offset from the center of rotation. The device can further include an actuator configured to cause spatial translation of the single objective lens, the actuator being a piezoagitator or a motor.

The illumination guide can be configured to spatially translate relative to the distal shaft between at least a first location and at least a second location a distance away from the at least a first location, the first location and the second location being at a subtended parallax angle. The device can be configured to capture images of the intraocular object as the illumination guide spatially translates to provide the three dimensional visualization. The spatial translation of the illumination guide can occur side-to-side from the at least a first location to the at least a second location. The spatial translation of the illumination guide can be rotational around the longitudinal axis from the at least a first location to the at least a second location. The device can further include an actuator configured to cause spatial translation of the illumination guide, the actuator being a piezoagitator or a motor.

The illumination guide can include a first illumination guide and a second illumination guide. The at least one objective lens can be aligned with the longitudinal axis of the distal shaft, the first illumination guide can be spatially off-set to a first side of the at least one objective lens, and the second illumination guide can be spatially off-set to a second side of the at least one objective lens. The device can be configured to capture images of the intraocular object as the first illumination guide illuminates a first spatial location of the object, the second illumination guide illuminates a second spatial location of the object, and the first and second guides simultaneously illuminate the object at the first spatial location and the second spatial location. The distal shaft can be configured to slide from a first position in which the first and second illumination guides are sheathed by the distal shaft to a second position in which the first and second illumination guides are unsheathed from the distal shaft. The first and second illumination guides can move outward away from one another when the distal shaft is in the second position. The device can further include a shape memory element configured to urge the first and second illumination guides to move outward from one another upon withdrawing the distal shaft into the second position.

The at least one objective lens can include a first objective lens and a second objective lens horizontally offset from the first lens. A distance between an optical axis of the first objective lens and an optical axis of the second objective lens can define a parallax angle for a distance from the first and second objective lenses to the intraocular object. A subtended parallax angle can form two images of the intraocular object at different angular perspectives. The first objective lens can be coupled to a first bundle of the at least one image transmitter and the second objective lens can be coupled to a second bundle of the at least one image transmitter. The at least one image sensor can include a first image sensor in optical communication with a proximal end of a first image transmitter and a second image sensor in optical communication with a proximal end of a second image transmitter. The at least one image sensor can be a single image sensor in optical communication with a proximal end of a first image transmitter and a proximal end of a second image transmitter. The distal shaft can be configured to slide from a first position in which the first and second objective lenses are sheathed by the distal shaft to a second position in which the first and second objective lenses are unsheathed from the distal shaft. The first objective lens and the second objective lens can move outward away from one another increasing the distance between their respective optical axes when the distal shaft is in the second position. The device can further include a shape memory element configured to urge the first and second objective lenses to move outward from one another upon withdrawing the distal shaft into the second position. The shape memory element can include a spring or a shape-set Nitinol component. The first and second objective lenses can be ball lenses coupled to the shape memory element. A maximum outer dimension of the first and second objective lenses in the second position can be greater than an inner diameter of the distal shaft. The device can provide monocular vision when the distal shaft is in the first position and the first and second objective lenses are sheathed. Images from the first and second objective lenses can be combined using stereo viewing technique. The stereo viewing technique can include anaglyph spectacles or a binocular combining system.

The at least one objective lens can be a single objective lens configured to spatially translate relative to the distal shaft between at least a first location and at least a second location a distance away from the at least a first location, wherein the spatial translation of the single objective lens can be along the longitudinal axis towards and/or away from the intraocular object from the at least a first location to the at least a second location. The device can be configured to capture images of the intraocular object as the single objective lens spatially translates to provide the three dimensional visualization. The images captured can be at different focal places along the intraocular object and configured to create a topographical overlay. The images captured at the different focal planes can be combinable to create at least one focus-stacked image. The device can further include an actuator configured to cause spatial translation of the single objective lens, the actuator being a piezoagitator or a motor.

The at least one objective lens can be a liquid lens configured to change focal length, and the device can be configured to capture images of an object as the focal length of the liquid lens changes. The at least one objective lens can be a ball lens or a liquid lens. The outer diameter of the distal shaft can be 0.020 inches to 0.072 inches. The second portion can be detachable from the first portion for reuse of the second portion and disposal of the first portion. The at least one image transmitter can comprise a stack of rode lenses or a fiber bundle. The device can further include a secondary illumination source. The secondary illumination source can contain red, green, or blue light for the purpose of photobiomodulation.

In an interrelated implementation, provided is an endoscopic device configured to provide three-dimensional visualization of an intraocular object. The device includes a first portion including a distal shaft having a lumen extending along a longitudinal axis, at least one image transmitter extending through the lumen of the distal shaft, and a first objective lens and a second objective lens horizontally offset from the first lens. The device includes a second portion coupled to the first portion, and the second portion includes a housing and at least one image sensor positioned within the housing in optical communication with a proximal end of the at least one image transmitter. The first objective lens is coupled to a first bundle of the at least one image transmitter and the second objective lens is coupled to a second bundle of the at least one image transmitter. The distal shaft is configured to slide from a first position in which the first and second objective lenses are sheathed by the distal shaft to a second position in which the first and second objective lenses are unsheathed from the distal shaft. The first objective lens and the second objective lens move outward away from one another increasing the distance between their respective optical axes when the distal shaft is in the second position.

In an interrelated implementation, provided is an endoscopic device configured to provide three-dimensional visualization of an intraocular object. The device includes a first portion including a distal shaft having a lumen extending along a longitudinal axis, at least one image transmitter extending through the lumen of the distal shaft, and an objective lens having an optical axis and positioned within the lumen. The device includes a second portion coupled to the first portion, and the second portion includes a housing and at least one image sensor positioned within the housing in optical communication with a proximal end of the least one image transmitter. The objective lens is configured to spatially translate relative to the distal shaft between at least a first location and at least a second location horizontally off-set from the first location. The device is configured to capture images of the intraocular object as the objective lens spatially translates to provide the three-dimensional visualization. A distance between the optical axis of the objective lens when positioned at the first location to the optical axis of the objective lens when positioned at the second location is proportional to a working distance of the endoscope such that the distance defines a parallax angle for a distance from the first and second locations to the intraocular object. A subtended parallax angle forms two images of the intraocular object at different angular perspectives.

In an interrelated implementation, provided is an endoscopic device configured to provide three-dimensional visualization of an intraocular object including a first portion including a distal shaft having a lumen extending along a longitudinal axis, a first illumination guide and a second illumination guide extending through the lumen of the distal shaft, at least one image transmitter extending through the lumen of the distal shaft, and at least one objective lens positioned within the lumen and aligned with the longitudinal axis of the distal shaft. The device includes a second portion coupled to the first portion, and the second portion includes a housing, at least one illumination source positioned within the housing in optical communication with a proximal end of the first illumination guide and a proximal end of the second illumination guide, and at least one image sensor positioned within the housing in optical communication with a proximal end of the at least one image transmitter. The first illumination guide is spatially off-set to a first side of the at least one objective lens and the second illumination guide is spatially off-set to a second side of the at least one objective lens. The device is configured to capture images of the intraocular object as the first illumination guide illuminates a first spatial location of the object, the second illumination guide illuminates a second spatial location of the object, and the first and second illumination guides simultaneously illuminate the object at the first spatial location and the second spatial location.

### BRIEF DESCRIPTION OF THE DRAWINGS

These and other aspects will now be described in detail with reference to the following drawings. Generally, the figures are not to scale in absolute terms or comparatively, but are intended to be illustrative. Also, relative placement of features and elements may be modified for the purpose of illustrative clarity.

FIG. 1 is a box diagram illustrating a 3D endoscope;

FIG. 2A is a schematic top view of an implementation of a three-dimensional (3D) endoscope;

FIG. 2B is a top view of a distal tip of the device of FIG. 2A;

FIG. 3 is a schematic top view of a distal end of an interrelated implementation of a 3D endoscope;

FIG. 4 is an end view of a translating lens for use with the device of FIG. 3;

FIG. 5 is an end view of a rotating lens for use with the device of FIG. 3;

FIG. 6 is a schematic side view of a distal end of an interrelated implementation of a 3D endoscope;

FIG. 7A is a schematic top view of an interrelated implementation of a 3D endoscope in a sheathed position;

FIG. 7B is a top view of the device of FIG. 7A in an unsheathed position; and

FIG. 8 is a schematic top view of an interrelated implementation of a 3D endoscope using a single image sensor.

It should be appreciated that the drawings are for example only and are not meant to be to scale. It is to be understood that devices described herein may include features not necessarily depicted in each figure.

### DETAILED DESCRIPTION

Disclosed is a handheld 3D endoscopic surgical device that can operate in the intraocular space of the eye or other regions and can be used to visualize the internal structures of the eye (e.g., trabecular meshwork in the anterior chamber, posterior chamber, retina in the vitreous chamber) or other regions in three dimensions. The endoscopic device can be used in other microsurgical applications outside of ophthalmology, including but not limited to the field of otology, pulmonology, otolaryngology, and intravascular neurosurgery. The endoscopic device may use Optical Coherence Tomography (OCT), visible light, non-visible light, or ultrasound for imaging. The endoscopic device may also emit light wavelengths used in low-level laser therapy, or photobiomodulation therapy, to induce photophysical and photochemical changes at targeted therapy sites.

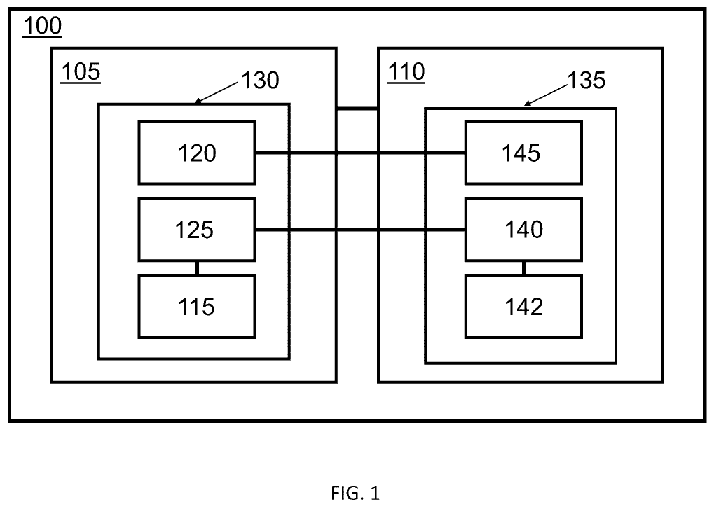

More particularly and as will be described in detail below, the endoscopic devices 100 described herein can have a distal portion 105 configured to be inserted, at least in part, into the eye coupled to a proximal portion 110 configured to remain outside the eye (see FIG. 1). The distal portion 105 can contain components such as one or more objective lenses 115, one or more illumination guides 120, one or more image transmitters 125, etc. that are contained within or extend through a lumen of a distal shaft 130. The lens(s) 115, guides 120, and image transmitters 125 can be entirely contained within the distal shaft 130 or at least a portion of which extends outside the distal shaft 130. For example, a proximal end region of the illumination guide 120 and/or image transmitter 125 may extend outside a proximal end of the distal shaft 130 for optical coupling with their respective component part within the proximal portion. The objective lens 115 focuses an image of the object on a distal end of the image transmitter 124. The image transmitter 125 can be a stack of rod lenses, a single fiber optic, or light transmitting fiberoptic bundles, or other element.

The proximal portion 110 can include a housing 135 that can contain one or more illumination sources 145 and one or more image sensors 140 of an imaging unit 142 to be connected to proximal end regions of the one or more image transmitters 125 within the housing 135. The housing 135 has a small form factor and can be formed as a handle for ergonomic use for manipulation by a user and thus, remains outside of the eye. The one or more image transmitters 125 transmit an image from the one or more lenses 115 to the one or more image sensors 140 in the housing. A distal end of the one or more image transmitters 125 is adjacent to the one or more lenses 115 and a proximal end of the one or more image transmitters 125 is adjacent to the one or more image sensors 140. The image sensor 140 may be, for example, a camera including a lens, CMOS, or imaging CCD.

The distal shaft 130 of the distal portion 105 projects distally from the housing 135 and is sized for insertion in an eye (e.g., through the cornea or sclera into the anterior chamber and/or the posterior chamber between the iris and the capsular bag or into the vitreous). The distal shaft 130 of the device 100 may have a diameter of 25 gauge (0.020 inches/0.5 mm) up to 15 gauge (0.072 inches/1.8 mm). The one or more lenses 115, the one or more illumination guides 120, one or more image transmitters 125 can be positioned within and/or extend through at least a portion of the distal shaft 130. In some implementations, the image sensor 140 can be positioned at a distal end of the shaft 130. In this implementation, the device need not incorporate any fiber optic bundle or rod lens as the image transmitter 125. Instead, an electronic connection or wire from the image sensor 140 at the tip can extend proximally through the shaft to the proximal portion 110 similar to “chip in the tip” technologies.

Again with respect to FIG. 1, the one or more illumination guides 120 are used to illuminate an object for proper visualization. The illumination guides 120 can direct one or more of visible light or non-visible light toward the object by transmitting illumination from one or more illumination sources 145 through the one or more illumination guides 120 to the object. The illumination guides 120 can also use single wavelength light or use multiple wavelengths of light. For example, all illumination guides 120 in a device may use the same wavelength light, or different illumination guides 120 may use different wavelengths of light. Proximal ends of the illumination guides 120 can be connected to one or more illumination sources 145 in the housing 135. Illumination sources 145 can be one or more of ultrasonic transducers or light emitters such as LEDs. The LEDs can emit various wavelengths of light, including white, red, near-infrared, blue, green, ultraviolet, or near ultraviolet light. Light can enter the proximal end of the illumination guides 120 and exit the distal ends to illuminate the object. In an alternative, the device can incorporate an ultrasound transducer as an alternative to light. The ultrasound transducer can be located near the distal tip of the device or can be present within the proximal portion and in communication with a waveguide tube extending through the distal shaft 130 to transmit the waves. The waveguide tube can have be similar in size to the wavelength of the ultrasound produced.

Again with respect to FIG. 1, the illumination guides 120 may be fiber optic light guides, reducing the diameter size so as to extend within the distal shaft 130 and reducing cost. As will be discussed in greater detail below, the components of the devices, such as the one or more illumination guides 120, the one or more lenses 115 have reduced size, which significantly reduces the size of the distal shaft 130 inserted into the eye while still providing improved imaging and 3D visualization. Small outer diameter of the distal shaft 130 is desired for improved healing after an incision is made in the cornea, but too small of a diameter interferes with proper 3D visualization. Three-dimensional imaging is desirable for certain applications in which volume of structures is important to understand that wouldn't otherwise be possible in two-dimensions, for example, to asses tissue growth, tumor size, and certain structures in which the volume is important (e.g., ciliary body). The 3D visualization allows for quantification of dimensions to understand also where in space tools are being navigated for depth perception. In small spaces, such as the anterior chamber, posterior chamber, and vitreous, this depth perception is desirable so as to avoid damaging delicate tissues and structures. The devices described herein provide effective three dimensional visualization in small spaces for at a cost that is low enough to allow for disposable parts. For example, the distal portion 105 and the proximal portion 110 of the devices described herein may reversibly coupled to one another such that the distal portion, which is designed to come into contact with patient fluids, can be disposed of after use and the proximal portion 110, which is designed to remain out of contact with patient fluids, can be reused.

Use of the terms “hand piece” “hand-held” or “handle” herein need not be limited to a surgeon's hand and can include a hand piece coupled to a robotic arm or robotic system or other computer-assisted surgical system in which the user uses a computer console to manipulate the controls of the instrument. The computer can translate the user's movements and actuation of the controls to be then carried out on the patient by the robotic arm.

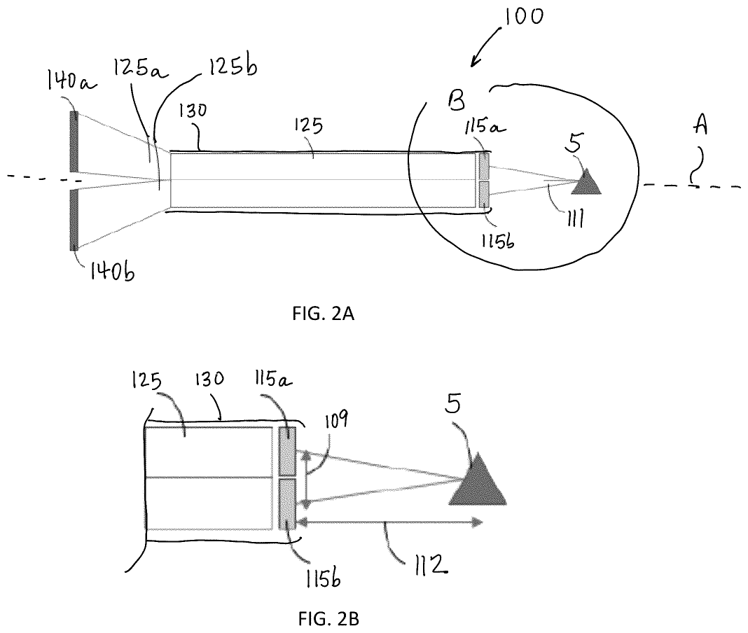

FIG. 2A is a schematic top view of an implementation of a 3D endoscopic device 100. FIG. 2B is a top view of a distal end region of the 3D endoscopic device 100 of FIG. 2A taken at circle B. As described with regard to FIG. 1 although not shown in FIGS. 2A-2B, the device 100 can include a distal portion 105 that generally includes the components of the device that are configured to come into contact with the patient that is coupled to a proximal portion 110 that generally includes the components of the device that are configured to remain outside the patient.

Still with respect to FIG. 2A, the image transmitter 125 is shown extending from a proximal end region of the device 100 toward a distal end region of the device 100. The image transmitter 125 can extend at least partially through the distal shaft 130, which has an outer diameter sized to be inserted at least partially within the patient as described elsewhere herein (e.g., 0.020″ to 0.072″ outer diameter). The distal end region of the image transmitter 125 can be operatively coupled to one or more lenses 115 and the proximal end region of the image transmitter 125 can be operatively coupled to one or more image sensors 140 of the imaging unit 142. In the implementation illustrated in FIGS. 2A-2B, the 3D endoscopic device 100 includes two lenses 115a, 115b coupled to distal end region of the image transmitter 125 and two image sensor 140a, 140b coupled the proximal end region of the image transmitter 125.

As discussed elsewhere herein, the distal shaft 130 is part of the distal portion 105 of the endoscopic device 100 and is designed to come into direct contact with the patient, such as through a corneal incision in the eye or through a scleral port device. In ophthalmology, smaller incisions are better due to their lower risk of leakage and/or infection and improved healing after surgery. Thus, the distal shaft 130 of the endoscopic devices 100 described herein are preferably no greater than 15 gauge (i.e., 0.072″ or about 1.8 mm in outer diameter). Additionally, the devices described herein preferably incorporate a distal portion 105 that can be disposed of after use such as by removably detaching the distal portion 105 from the proximal portion 110 as described in more detail below. It is therefore preferred to minimize the size and also the cost of the component parts of the distal portion 105. As such, the one or more lenses 115 may be any lens that can be sized appropriately to fit within the inner diameter of the distal shaft 130 that is also relatively inexpensive such that the distal portion 105 can be disposed of after a single use. The one or more objective lenses 115 can include lenses such as spherical ball lenses, liquid lenses, conventional multi-element lenses or doublets with an aperture. The objective lenses 115 are preferably no greater than about 2 mm, no greater than about 1 mm, no greater than about 0.9 mm, no greater than about 0.8 mm, no greater than about 0.7 mm, no greater than about 0.6 mm, no greater than about 0.6 mm. If more than one lens 115a, 115b is to be contained within the distal shaft 130, the width of the objective lens 115 is less than about half the inner diameter of the shaft 130 so as to be contained within the distal shaft 130. The lenses 115a, 115b can be designed to have an outer diameter such that both lenses 115a, 115b can fit side-by-side within the distal shaft 130. For example, each lens 115a, 115b can have a diameter within the range of 0.010″ to 0.036″ (0.01 mm-0.9 mm), depending on the diameter of distal shaft 130. Lenses 115a and 115b can have the same or similar diameter. Lenses 115a, 115b can be spherical ball lenses, which can be sized appropriately small while remaining relatively inexpensive, reducing cost and allowing the distal portion 105 of the device 100 to be disposed of after a single use.

The lenses 115a, 115b can be horizontally offset from each other by a distance 109. The distance 109 is between the optical axis of the first lens 115a to the optical axis of the second lens 115b (see FIG. 2B). The distance 109 is proportionate to a working distance (the distance from the lens to the focal plane) of the endoscope 100 and is typically between 0.10 mm and 1 mm (0.004″ and 0.039″). The distance 109 between the optical axes of the two lenses 115a, 115b define a parallax angle 111 for any given distance 112 from the lenses 115a, 115b to the object 5 being viewed. An appropriately subtended parallax angle 111 will result in two images of the object 5 that have slightly different angular perspectives. For example, a parallax angle 111 that is too small will not have large enough differences in angular perspectives, and the depth information gained will not generate detailed 3D visualization. A parallax angle 111 that is appropriately sized will be large enough to convey detailed depth information for improved 3D visualization. The device 100 may have a fixed distance 109 between the lenses 115a, 115b, determining the working distance of the endoscope 100.

The width of the lenses is directly related to how big the object to be viewed is and how small the working distance is. If the lens 115 is not wider than the object or the distance between two lenses 115a, 115b is not greater than a width of the object, the 3D visualization is impaired. The size of the object being visualized in 3D is driven by how wide the lenses can be and/or how far apart the lenses can be placed apart. For example, two lenses 115 placed about 0.45 mm apart allow for viewing of the ciliary body surface relief, but likely unhelpful in viewing the edges of the ciliary body. In contrast, two lenses 115 placed about 0.45 mm apart is suitable to visualize surface features of the retina that are smaller than about 1 mm. The larger the object to be viewed, it is preferable to increase the separation of the lenses and/or increase the working distance between the lens and the object. The separation between the lenses can be maximized without being constrained by the small distal shaft size, for example, by allowing for active expansion of lenses outside the distal shaft, which will be described in more detail below.

The proximal end region of the image transmitter 125 can be split into two separate image transmitters 125a, 125b. A first image transmitter 125a transmits a first image of the object 5 from the first lens 115a and a second image transmitter 125b transmits a second image of the object 5 from the second lens 115b. The first and second images from the first and second lenses 115a, 115b are transmitted by the portions of the image transmitters 125a, 125b onto corresponding image sensors 140a, 140b (see FIG. 2A). Unlike in conventional endoscope devices, which either only produce a monocular image or are able to produce a 3D image but are not sized appropriately small for intraocular use, the device 100 of FIGS. 2A-2B is able to produce intraocular 3D images.

The two images from the image sensors 140a, 140b of the imaging unit 142 can be combined using an appropriate stereo viewing technique, including but not limiting to anaglyph spectacles or a binocular combining system, or can be combined using a computer algorithm to form a 3D image or topographic map. Combined images may also be viewed on a monitor or VR/AR goggles. The housing 135 may contain a processor that performs the processing required for the stereo viewing technique or image combination technique, or the housing 135 may be connected to an external device that contains the processor. The connection between the housing 135 and the external device may be a wired connection or a wireless connection.

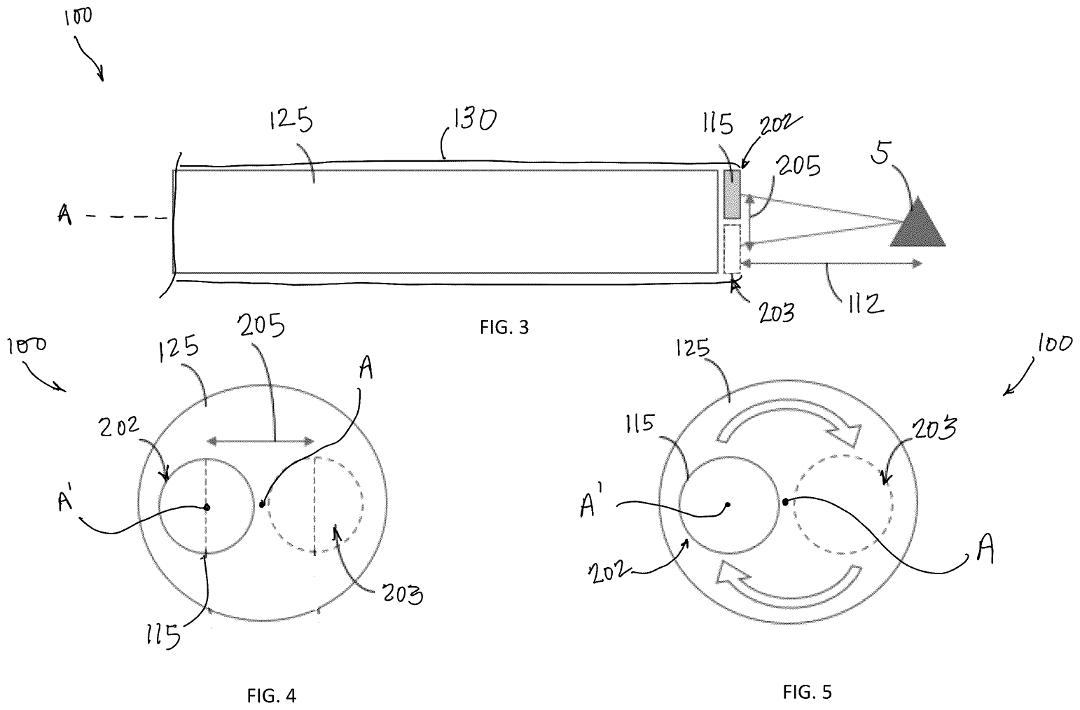

In some implementations, such as that shown in FIGS. 3-5, the endoscopic device 100 can include a single objective lens 115. In some implementations, the single lens 115 can be moved spatially, such as linearly or rotationally translated, between different positions to act as virtual multiple lenses. As in the implementations described above, the device 100 can include a distal portion 105 and a proximal portion 110 incorporating one or more of the components in each as discussed above with regard to FIGS. 1, 2A-2B. Details of all the components are not repeated herein for each figure for the sake of brevity. It should be appreciated that each of the implementations of the endoscopes described herein can include any of a variety of combination of components.

FIG. 3 is a schematic of the device 100 showing the image transmitter 125 having a distal end region arranged relative to a single objective lens 115. The lens 115 is located horizontally off-set from the longitudinal axis A of the distal shaft 130. The lens 115 can be moved between at least two locations, a primary translation position 202 and a secondary translation position 203. The device in FIGS. 2A-2B incorporates two separate lenses 115a, 115b at the two locations with a distance 109 between the optical axes of the two lenses 115a, 115b. The single objective lens 115 of the device in FIG. 3 is moved between the two translation positions 202, 203. The optical axis A′ of the single objective lens 115 when at the primary translation position 202 is a distance 205 away from the optical axis A′ of the single objective lens 115 when moved to the secondary translation position 203.

The single objective lens 115 may be translated between the two positions 202, 203 side-to-side along a horizontal plane as shown in FIG. 4. The central axis A′ of lens 115 can be moved horizontally the translation distance 205 between the primary translation position 202 and the secondary translation position 203. Alternatively, the lens 115 may be translated rotationally as shown in FIG. 5. The lens 115 can be rotated around the longitudinal axis A of the shaft 130 between the primary translation position 202 and the secondary translation position 203. The rotational translation of the lens 115 around the longitudinal axis A of the shaft 130 can result in a number of rotational positions beyond just the primary 202 and secondary positions 203. Offsetting the lens 115 from the center of rotation will allow the lens 115 to spatially translate when rotated.

Horizontal translation of lens 115 can be caused by, for example, a piezoagitator, a motor, or another linear actuator. The lens 115 can be engaged with the piezoagitator such that the vibrational side to side movement of the piezoagitator can be translated to the lens 115, causing the lens 115 to vibrate side to side. The lens 115 can be engaged with the piezoagitator through an intervening element, such as a shaft or rod connecting the two. The frame rate of image capture can align with the frequency of vibration such that images are captured at the positional extremes of translation.

Rotational translation of lens 115 can be caused by a rotating motor, a piezoagitator, or another rotational actuator. The piezoagitator can be engaged with the lens 115 and rotational motion mechanisms such that vibrational movement of the piezoagitator is translated into rotational motion of the lens 115. The rotational motion mechanism may include a ratchet and pawl mechanism. The frame rate of image capture can align with the rotational speed such that images are captured at the positional extremes of translation.

Images can be viewed and/or recorded while the lens 115 is at the positional extremes along the translation path to create multiple angular perspectives of an object 5. The positional extremes can vary proportionally to the magnitude of the translation distance 205 and the working distance 112 between the lens 115 and the object 5 being viewed. Incorporating a single lens 115 within the distal shaft 130 can reduce cost of the device. The type of lens 115 can also reduce cost. The lens 115 can be a small spherical ball lens as described elsewhere herein.

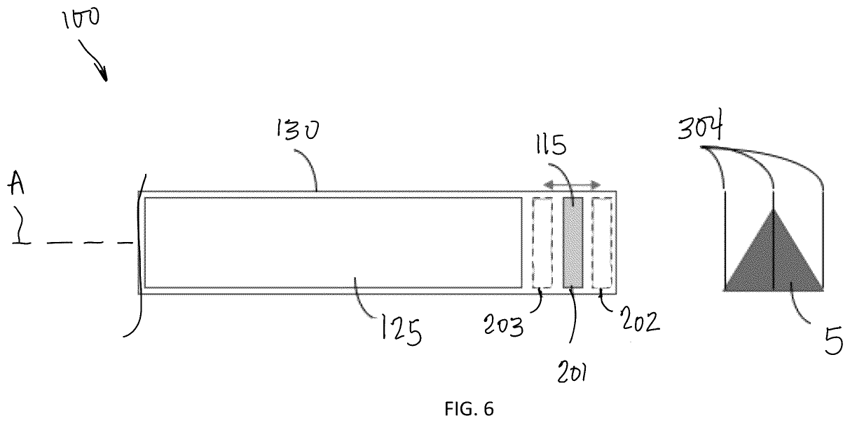

In some implementations, the single lens 115 of the device 100 can also be translated between various spatial positions along the longitudinal axis A of the distal shaft 130 (see FIG. 6) to achieve three dimensional viewing with a single lens 115. The endoscopic device 100 can be used to create a topographical overlay utilizing multiple frames recorded at varying distances from the lens 115 to the object 5. The lens 115 has a finite depth of field (defined here as a lens 115 having a high contrast focal point over a very small distance from the object 5 relative to the size of the object). Image frames can be recorded at different distances from the object 5 along the longitudinal axis A of the distal shaft 130. The lens 115 can be moved axially along the axis A to achieve at least a primary position 202 and a secondary position 203 from the object 5, to capture multiple images at various focal planes 304 along the object 5. FIG. 6 shows three translation positions including an initial position 201, a primary position 202 that is closer to the object 5 than when in the initial position 201, and a secondary position 203 that is further away to the object 5 than when in the initial position 201. The different axial positions of the single lens 115 create multiple focal planes 304 of the object 5. The images can be transferred to an image sensor (not shown in FIG. 6) by use of the image transmitter 125 as described elsewhere herein.

Images are captured having a region of higher contrast and lower contrast. Once the images are captured at the plurality of focal planes 304, the stacked images can be combined into a focus-stacked image. The high contrast portions of each image are combined to create an overall high contrast image. Since each high contrast region is associated with a specific known lens-to-object distance, each high contrast region comprising the overall high contrast image can be coded visually to indicate topographic (depth) information. Such coding can incorporate colored or spaced lines indicating relief as seen on a topographic map. As described above, a processor of an external device or contained within housing 135 can process the images into a topographic map.

The spatially translating lens 115 can be a lens with a fixed focal length, such as a ball lens or another type of objective lens. However, in some implementations, the single lens 115 of the device 100 need not translate spatially relative to the longitudinal axis A of the distal shaft 130 to achieve the above-described effect. The lens 115 can be a liquid lens having a focal length configured to change so as to capture multiple images at various focal planes 304 along the object 5. The images may be combined similarly as described above. For example, a lens 115 that is a liquid lens can be electronically controlled to change shape and thereby change focal length. Different focal lengths will create multiple focal planes 304 of the object 5, and images can be captured as described above. Electronic control over the focal length of the liquid lens may be achieved by acousto-optical tuning, shape-changing polymers, dielectric actuation, application of magnetic fields, or other suitable methods.

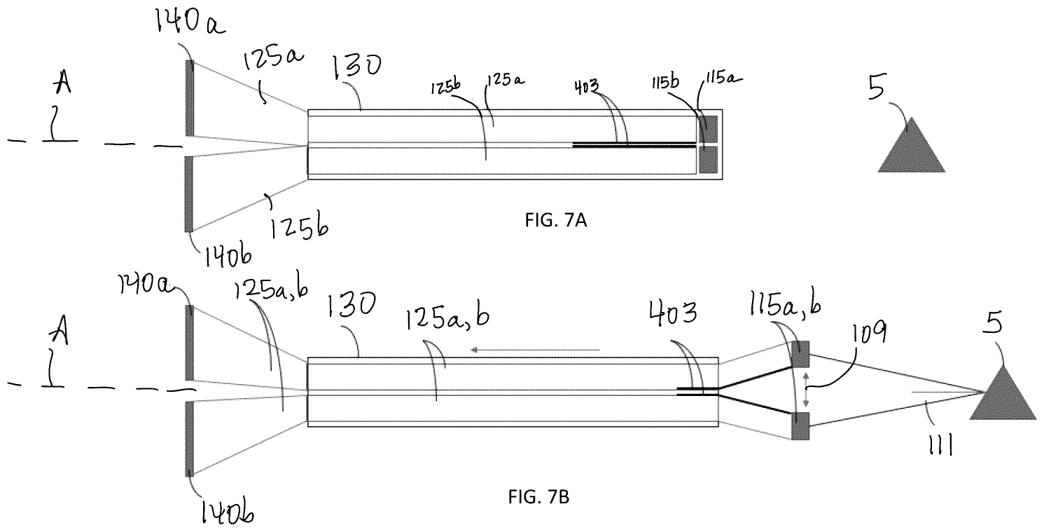

The lenses of the devices described above are restricted by the inner lumen size of the distal shaft 130. In a further implementation, the lens 115 can be moved outside the inner diameter of the distal shaft 130 to increase the distance between the viewpoints of the object. FIGS. 7A-7B shows the device 100 having two lenses 115a, 115b that are configured to move outward away from each other so that the distance between them is greater than an inner diameter of the distal shaft 130. Each of the lenses 115a, 115b can be attached at or adjacent to a distal tip of an extending armature that can be extended laterally to create a specific angle between each lens 115a, 115b and the object 5. FIG. 7A shows the device 100 in a sheathed position, in which the distal shaft 130 contains the lenses 115a, 115b within the lumen of the shaft 130. The lenses 115a, 115b are adjacent to one another so that a distance 109 between their respective optical axes is minimized and constrained by the inner diameter of the distal shaft 130. FIG. 7B shows the device 100 in an unsheathed position, in which the lenses 115a, 115b are located outside the lumen of the distal shaft 130 and splayed away from one another. The distance between their optical axes is maximized and unconstrained by the inner diameter of the distal shaft 130. The increased distance between them allow for each lens 115a, 115b to have a different perspective view of the object 5 and thus form a larger parallax angle 111 between the lenses 115a, 115b and the object 5 than if the lenses 115a, 115b were sheathed within and constrained by the distal shaft 130.

The endoscopic device 100 can be inserted into the eye while in the sheathed position and moved into an unsheathed position while inside the eye. The distal shaft 130 may be moved proximally and retracted to uncover the lenses 115a, 115b to thereby transition the device from the sheathed position to the unsheathed position, and can be advanced in a distal direction to transition the device from the unsheathed position to the sheathed position. Alternatively, the lenses 115a, 115b may be moved distally and advanced out of the distal shaft 130 to thereby transition from the sheathed position to the unsheathed position, and can be moved proximally to retract back into the distal shaft 130 and return to the sheathed position. Transitioning from the sheathed position to the unsheathed position releases the lenses from constraint of the distal shaft 130 so that they can be urged outward apart from each other.

Actuation of lens 115a, 115b movement or distal shaft 130 movement can be controlled by various user inputs, such as buttons, sliders, an interactive display, or other suitable inputs.

A shape memory element or armature 403 can be incorporated that is designed to urge and displace the lenses 115a, 115b outwards away from one another once unsheathed by the distal shaft 130. Each extending armature 403 can include an image transmitter 125 to transmit the captured image from each lens 115a, 115b. The shape memory element 403 can include one or more springs or shape-set Nitinol components that splay outward when unconstrained. The outward motion of the lenses 115a, 115b away from the longitudinal axis A of the shaft due to the shape memory elements 403 increases the distance 109 between optical axes of the lenses 115a, 115b than normally would be available for a given inner diameter of the distal shaft 130, thus improving three dimensional visualization in narrow endoscopes and allowing the device 100 to have a small diameter. The distal shaft 130 is limited in outer diameter to be no greater than about 15 gauge, or no greater than about 20 gauge, or no greater than about 25 gauge, or no greater than about 2.2 mm so as to be useful in small spaces of the eye and for insertion through an opening of the eye that is less likely to leak and/or need incisions for closure. The sheathed maximum outer dimension of the lenses 115a, 115b can be minimized to be inserted through small incisions while the unsheathed maximum outer dimension of the lenses 115a, 115b can be maximized to provide improved three dimensional visualization. The unsheathed maximum outer dimension of the lenses 115a, 115b can be in the range of 0.04″ to 0.12″ for a 25 gauge distal shaft 130, or a range of 0.07″ to 0.21″ for a 20 gauge distal shaft 130, or 0.14″ to 0.42″ for a 15 gauge distal shaft 130. When the lenses 115a, 115b are too close together, the parallax angle is small and the images from each lens do not show significant displacement of features of object 5. Feature displacement encodes depth information in stereoscopic images, and images that are too similar with little feature displacement do not allow for good 3D visualization, instead creating monocular visualization when the lenses are close to each other in the sheathed position. Increasing the distance between lenses 115a, 115b increases the parallax angle, which causes increased displacement of features of object 5 shown on each image. The increased displacement allows for enhanced depth information and 3D visualization.

Several implementations described above (e.g., FIGS. 3, 4, 5, 6, and 7A-7B) incorporate one or more objective lenses 115 that are configured to move relative to the shaft 130 so as to provide enhanced 3D visualization. However, the lens(es) 115 need not move relative to the shaft 130 to achieve 3D visualization. FIGS. 2A-2B shows an implementation of a device having two lenses 115 that are off-set from the central axis A of the shaft 130 and remain fixed during use while still providing 3D visualization. The device can also incorporate a single objective lens 115 that remains fixed relative to the distal shaft 130 during use and 3D visualization is provided by off-axis illumination. As an example, instead of the device incorporating two objective lenses 115a, 115b coupled so as to splay outward as shown in FIGS. 7A-7B, two illumination guides 120 can be arranged relative to the shape memory element 403 so that upon being unsheathed the guides 120 splay outward relative to a single objective lens 115 fixed at a distal end region of the shaft 130. The lens 115 remains fixed, for example, aligned with the central axis A of the shaft 130 and the guides 120 splay outward relative to the lens 115, for example, to either side of the lens 115. The illumination guides 120 are positioned at different spatial positions that are off-set relative to the longitudinal axis A of the distal shaft 130. The shadow and surface highlight information in the captured images can be used to form a three dimensional image of the object 5. The width between the splayed guides 120 can vary, for example, the distance between the guides 120 can be increased to, in turn, increase the possible object coverage, if desired.

The off-set illumination need not incorporate two illumination guides 120. Instead, a single illumination guide 120 and a single objective lens 115 can be incorporated. The lens 115 can remain fixed and the single illumination guide 120 translate between at least two positions such that illumination of the object 5 occurs at different spatial positions off-set relative to the longitudinal axis A of the distal shaft 130 and to the lens 115. This provides the effect of spatially translation the lens 115 without needing to actually move the lens. Multiple images can be taken of the object 5 with the lens 115 that remains stationary while the illumination guide 120 is translated linearly or rotationally, such as described above with regard to FIGS. 3-5. Variation of illumination of the object 5 occurs with each captured image. The shadow and surface highlight information in the captured images can be used to form a three dimensional image of the object 5. The device can be arranged to undergo outward splay as described above with regard to FIGS. 7A-7B such that the single illumination guide 120 can move between different positions relative to the longitudinal axis A that are off-set by a greater translation distance that would otherwise be allowed with a small distal shaft inner diameter.

In still another implementation, light can be directed toward the object 5 from an illumination guide 120 that is offset from the central axis A of the shaft. Neither the guide 120 nor the lens 115 are moved relative to the distal shaft to achieve the effect of different spatial positions. For example, the objective lens 115 can be positioned at a distal end of the distal shaft 130 between a first illumination guide 120 off-set from the lens 115 and a second illumination guide 120 off-set from the lens 115. The two illumination guides 120 can be in optical communication with one or more light sources configured to direct light through the guides 120. The light sources are activated according to a desired sequence to direct light through the guide 120 to the distal end of the shaft 130, for example, the light source for the first illumination guide 120 to the left of the lens 115 can be activated and deactivated, the light source for the second illumination guide 120 to the right of the lens 115 can be activated and deactivated, then the light sources for both the first and second illumination guides 120 can be activated and deactivated. The light projected off-axis relative to the lens 115 emphasizes the depth features on the side of the respective off-set illumination guide 120. When the object is illuminated through both illumination guides 120 the depth emphasis is eliminated and a baseline image can be achieved. An image can be taken for each illumination in the sequence and an algorithm used to combine the three images (i.e., left-side illuminated, right-side illuminated, both-sides illuminated) and extract depth information.

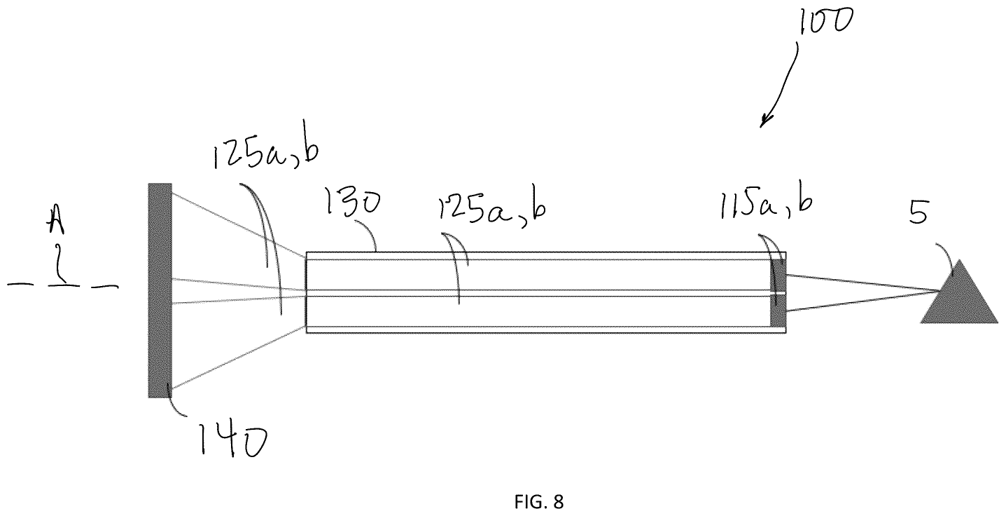

The image transmitter 125 of the device can be two separate image transmitters that can be split at a distal end region (see FIGS. 7A-7B) and/or a proximal end region into two separate image transmitters including 125a, 125b (see FIGS. 2A-2B, 7A-7B, and 8). The proximal end region of the separated image transmitters 125a, 125b can project onto one or more image sensors 140 of the imaging unit 142, and then images combined to create a 3D viewable image set that can be viewed by a surgeon by use of an appropriate viewing technique as described above. For example, FIGS. 2A-2B and 7A-7B shows two image sensors 140a, 140b at the proximal end region of the separated image transmitters 125a, 125b. The endoscopic device 100 can incorporate a single image sensor 140 for the capture of multiple images from the separated image transmitters 125a, 125b (see FIG. 8). The image transmitter 125 can be magnified and projected onto a single image sensor 140. There is a useful amount of “over-sampling” of image bundle fibers that allows for useful digital post-processing of the image. There remains a great deal of unused capacity (i.e., unused pixels) on the image sensor. The technology depicted by FIG. 8 illustrates multiple images generated by multiple lenses 115a, 115b projected on the same imaging sensor 140, which can be separated digitally. In the case of a stereo endoscope system having two or more optical imaging paths, as described above, both images can be projected onto a single sensor 140, using more of the available sensor pixels. The image projected onto the single image sensor 140 by the fiber bundle is a tiny fraction of total area of the image sensor 140. Thus, two images from two different fiber bundles can be projected onto the single sensor so that there is no overlap. The images can be extracted digitally and dealt with as individual images.

Regardless the implementation of the device, the objective lens 115, whether fixed or movable, or whether a single lens 115 or multiple lese are incorporated, can vary in configuration. Generally, the lens 115 is small enough to fit within the inner lumen of the distal shaft 130, which in turn, is sized to be inserted within a confined space, such as within the anterior chamber, the posterior chamber, or the vitreous cavity of the eye. The objective lens 115 includes lenses such as spherical ball lenses, liquid lenses, conventional multi-element lenses or doublets with an aperture. The objective lenses 115 are preferably no greater than about 2 mm, no greater than about 1 mm, no greater than about 0.9 mm, no greater than about 0.8 mm, no greater than about 0.7 mm, no greater than about 0.6 mm, no greater than about 0.6 mm. If more than one lens 115a, 115b is to be contained within the distal shaft 130, the width of the objective lens 115 is less than about half the inner diameter of the shaft 130 so as to be contained within the distal shaft 130. A ball lens can be sized appropriately small while remaining relatively inexpensive, reducing cost and allowing the distal portion 105 of the device 100 to be disposed of after a single use. The material of the ball lens as well as any of the lenses described herein can vary. Example materials of the lenses are BK7, fused silica, magnesium fluoride, zirconia, sapphire, ruby, etc, or any optical material including optical polymers. Sapphire, in particular, is a suitable material for any of the lenses described herein due to its ability to withstand heating to high temperatures without melting and cooling again. Sapphire also has a very high refractive index that is able to bend light well.

The image sensor 140 of any of the implementations of the devices described herein can vary, such as an imaging CCD or CMOS sensor. The image sensor 140 can be positioned within the housing 135 so as to receive an endoscope image from the one or more image transmitters 125 within the distal shaft 130. Image sensor 140 can be positioned away from the proximal end of the image transmitter 125 and receives the image. The device can additionally incorporate a glass window (e.g., a sapphire window) or other optically transparent element at a distal end region of the housing 135 that is configured to seal the housing 135 and prevent introduction of contaminants from the environment. The image sensor 140 is positioned within the housing 135 of the proximal portion 110 so as to receive an image from the image transmitter (e.g., fiberoptic bundle) within the distal shaft 130 of the distal portion 105.

The distal portion 105 and the proximal portion 110 of any of the implementations of the devices described herein can reversibly couple using a variety of mechanisms, such as a spring-load, magnetic attachment, screw-on, and clip-on, among other suitable mechanisms. This allows internal portion 105 to be disposable without significant expense, as opposed to reusable. External portion 110 of device 100 can be reused and thus requires sterilization. The design of internal portion 105 does not require it to withstand sterilization, which reduces cost and enhances ease of use. Coupling between reusable and disposable portions of the device can be achieved as described in U.S. patent application Ser. No. 18/072,389, filed Nov. 30, 2022, which is incorporated herein in its entirety.

Any of the implementations of the devices described herein can incorporate secondary or additional illumination sources 145 within the housing 135 of the proximal portion 110, such as an illumination source 145 containing red, green, or blue light for the purpose of photobiomodulation. The secondary illumination source 145 can be an LED that emits white, red (600-700 nm), near-infrared (770-1200 nm or 808 nm), blue, green, ultraviolet or near ultraviolet, or other colors of light. The secondary illumination source 145 can be optically coupled to a corresponding illumination guide 120 such that the light is transmitted into the intraocular space for photobiomodulation.

As mentioned above, endoscopic device 100 may connect to an external device for image processing. The external device may have a video monitor. The connection can include a wired communication port on housing 135 such as a RS22 connection, USB, Fire wire connections, proprietary connections, or any other suitable type of hard-wired connection configured to receive and/or send information to an external computing device. The endoscopic device 100 can also include a wireless communication port such that information can be fed between the device 100 and the external computing device via a wireless link, for example, to display information in real-time on the external computing device about operation and/or control programming of the device 100. It should be appreciated that the external computing device, such as a console or handheld device such as a tablet, can communicate directly to the device 100. Any of a variety of adjustments to and programming of the device can be performed using the external computing device. The wireless connection can use any suitable wireless system, such as Bluetooth, Wi-Fi, radio frequency, ZigBee communication protocols, infrared, or cellular phone systems, and can also employ coding or authentication to verify the origin of the information received. The wireless connection can also be any of a variety of proprietary wireless connection protocols.

In various implementations, description is made with reference to the figures. However, certain implementations may be practiced without one or more of these specific details, or in combination with other known methods and configurations. In the description, numerous specific details are set forth, such as specific configurations, dimensions, and processes, in order to provide a thorough understanding of the implementations. In other instances, well-known processes and manufacturing techniques have not been described in particular detail in order to not unnecessarily obscure the description. Reference throughout this specification to “one embodiment,” “an embodiment,” “one implementation, “an implementation,” or the like, means that a particular feature, structure, configuration, or characteristic described is included in at least one embodiment or implementation. Thus, the appearance of the phrase “one embodiment,” “an embodiment,” “one implementation, “an implementation,” or the like, in various places throughout this specification are not necessarily referring to the same embodiment or implementation. Furthermore, the particular features, structures, configurations, or characteristics may be combined in any suitable manner in one or more implementations.

The use of relative terms throughout the description may denote a relative position or direction. For example, “distal” may indicate a first direction away from a reference point. Similarly, “proximal” may indicate a location in a second direction opposite to the first direction. The reference point used herein may be the operator such that the terms “proximal” and “distal” are in reference to an operator using the device. A region of the device that is closer to an operator may be described herein as “proximal” and a region of the device that is further away from an operator may be described herein as “distal”. Similarly, the terms “proximal” and “distal” may also be used herein to refer to anatomical locations of a patient from the perspective of an operator or from the perspective of an entry point or along a path of insertion from the entry point of the system. As such, a location that is proximal may mean a location in the patient that is closer to an entry point of the device along a path of insertion towards a target and a location that is distal may mean a location in a patient that is further away from an entry point of the device along a path of insertion towards the target location. However, such terms are provided to establish relative frames of reference, and are not intended to limit the use or orientation of the devices to a specific configuration described in the various implementations.

As used herein, the term “about” means a range of values including the specified value, which a person of ordinary skill in the art would consider reasonably similar to the specified value. In aspects, about means within a standard deviation using measurements generally acceptable in the art. In aspects, about means a range extending to +/−10% of the specified value. In aspects, about includes the specified value.

While this specification contains many specifics, these should not be construed as limitations on the scope of what is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results. Only a few examples and implementations are disclosed. Variations, modifications and enhancements to the described examples and implementations and other implementations may be made based on what is disclosed.

In the descriptions above and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.”

Use of the term “based on,” above and in the claims is intended to mean, “based at least in part on,” such that an unrecited feature or element is also permissible.

The systems disclosed herein may be packaged together in a single package. The finished package would be sterilized using sterilization methods such as Ethylene oxide or radiation and labeled and boxed. Instructions for use may also be provided in-box or through an internet link printed on the label.

All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of any claims. No language in the specification should be construed as indicating any non-claimed element essential to the practice of the invention.

Groupings of alternative elements, embodiments, or implementations disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

### P EMBODIMENTS

P Embodiment 1. An ocular endoscopic system comprising: an imaging sensor; a means of image transmission from objective lens through a lumen to the camera lens (e.g. a fiber bundle); a light source; a plurality of objective lenses offset by a distance proportional to the working distance of the endoscope such that the distance between the optical axes of the two lenses define a parallax angle for any given distance from the object.

P Embodiment 2. An ocular endoscopic system comprising: an imaging sensor; a means of image transmission from objective lens through a lumen to the camera lens (e.g. a fiber bundle); a light source; one objective lens translated spatially for image capture in a plurality of locations. The images captured at each translation extrema will have different angular perspectives of the subject that can be used to create 3d image.

P Embodiment 3. The endoscopic system in P Embodiment 2 where the translation shift is achieved by a linear translation.

P Embodiment 4. The endoscopic system in P Embodiment 2 where the translation shift is achieved by a rotational motion where the lens is offset from the center of rotation.

P Embodiment 5. An ocular endoscopic system comprising: an imaging sensor; a means of image transmission from objective lens through a lumen to the camera lens (e.g. a fiber bundle); a light source; a lens system with a finite depth of field where images are captured within a plurality of distances from the subject, where each image captured is associated with a certain object distance from the subject; each image captures will have a region of higher contrast and lower contrast; the high contrast portions of each image will be combined to create an overall high contrast image. Those skilled in the art call this “focus stacking”. Since each high contrast region is associated with a specific known objective-to-subject distance, each high contrast region comprising the overall high contrast image can be coded visually to indicate topographic (depth) information. Such coding can be comprised of or colored or spaced lines indicating relief as seen on al topographic map.

P Embodiment 6. An ocular endoscopic system comprising: an imaging sensor; a means of image transmission from objective lens through a lumen to the camera lens (e.g. a fiber bundle); a light source; multiple extending lens armatures that can be extended laterally to create a specific angle between each lens and the subject.

P Embodiment 7. The endoscopic system in P Embodiment 6 where the extending lens armatures comprise an image transferring fiber bundle and an objective lens located at or near the distal end of each armature.

P Embodiment 8. The endoscopic system in P Embodiment 6 where images of the subject are captured through each of the optical systems associated with each extended armature.

P Embodiment 9. The endoscopic system in P Embodiment 6 where the distance between the extended armatures and associated optical components will define a parallax angle for any given objective to subject distance. Images recorded with the appropriate amount of parallax can be combined using an appropriate stereo viewing techniques such as anaglyph spectacles or a binocular combining system.

P Embodiment 10. The endoscopic system in P Embodiment 6 where the image created by the system is monocular when the armatures are not extended.

P Embodiment 11. A method of image capture in which a single image sensor is used to capture multiple images within the same frame and separated digitally to create multiple digital images.

P Embodiment 12. The method in P Embodiment 11 where a stereo endoscopic system, comprised of two or more optical image paths, is projected onto one sensor and used to create a 3d image.

P Embodiment 13. The endoscopic system of P Embodiment 2 where a secondary light source is employed containing red, green, or blue light for the purpose of photobiomodulation.

P Embodiment 14. The endoscopic system of P Embodiment 3 where a secondary light source is employed containing red, green, or blue light for the purpose of photobiomodulation.

P Embodiment 15. The endoscopic system of P Embodiment 5 where a secondary light source is employed containing red, green, or blue light for the purpose of photobiomodulation.

P Embodiment 16. The endoscopic system of P Embodiment 6 where a secondary light source is employed containing red, green, or blue light for the purpose of photobiomodulation.

P Embodiment 17. An endoscopic system comprising: an imaging sensor; a means of image transmission from objective lens through a lumen to the camera; a light source; a plurality of objective lenses offset by a distance proportional to the working distance of the endoscope such that the distance between the optical axes of the two lenses define a parallax angle for any given distance from the object.

P Embodiment 18. The device of P Embodiment 17 where the image transmission from the objective lens to the camera is achieved by a stack of rod lenses.

P Embodiment 19. The device of P Embodiment 17 where the image transmission from the objective lens to the camera is achieved by a fiber bundle.

P Embodiment 20. An endoscopic system comprising: an imaging sensor; a means of image transmission from objective lens through a fiber bundle; a light source; one objective lens translated spatially for image capture in a plurality of locations. The images captured at each translation extrema will have different angular perspectives of the subject that can be used to create 3d image.

P Embodiment 21. The endoscopic system in P Embodiment 20 where the translation shift is achieved by a linear translation.

P Embodiment 22. The endoscopic system in P Embodiment 20 where the translation shift is achieved by a rotational motion where the lens is offset from the center of rotation.

P Embodiment 23. An endoscopic system comprising: an imaging sensor; a means of image transmission from objective lens through a lumen to the camera lens (e.g. a fiber bundle); a light source; a lens system with a finite depth of field where images are captured within a plurality of distances from the subject, where each image captured is associated with a certain object distance from the subject; each image captured will have a region of higher contrast and lower contrast; the high contrast portions of each image will be combined to create an overall high contrast image. Those skilled in the art call this “focus stacking”, since each high contrast region is associated with a specific known objective-to-subject distance, each high contrast region comprising the overall high contrast image can be coded visually to indicate topographic (depth) information. Such coding can be comprised of or colored or spaced lines indicating relief as seen on a topographic map.

P Embodiment 24. An endoscopic system comprising: an imaging sensor; a means of image transmission from objective lens through a lumen to the camera lens (e.g. a fiber bundle); a light source; multiple extending lens armatures that can be extended laterally to create a specific angle between each lens and the subject.

P Embodiment 25. The endoscopic system in P Embodiment 24 where the extending lens armatures comprise an image transferring fiber bundle and an objective lens located at or near the distal end of each armature.

P Embodiment 26. The endoscopic system in P Embodiment 24 where images of the subject are captured through each of the optical systems associated with each extended armature.

P Embodiment 27. The endoscopic system in P Embodiment 24 where the distance between the extended armatures and associated optical components will define a parallax angle for any given objective to subject distance. Images recorded with the appropriate amount of parallax can be combined using an appropriate stereo viewing techniques such as anaglyph spectacles or a binocular combining system.

P Embodiment 28. The endoscopic system in P Embodiment 24 where the image created by the system is monocular when the armatures are not extended.

P Embodiment 29. A method of image capture in which a single image sensor is used to capture multiple images within the same frame and separated digitally to create multiple digital images.

P Embodiment 30. The method in P Embodiment 29 where a stereo endoscopic system, comprised of two or more optical image paths, is projected onto one sensor and used to create a 3d image.

P Embodiment 31. The endoscopic system of P Embodiment 17 where a secondary light source is employed containing red, green, or blue light for the purpose of photobiomodulation.

P Embodiment 32. The endoscopic system of P embodiment 20 where a secondary light source is employed containing red, green, or blue light for the purpose of photobiomodulation.

P Embodiment 33. The endoscopic system of P Embodiment 23 where a secondary light source is employed containing red, green, or blue light for the purpose of photobiomodulation.

P Embodiment 34. The endoscopic system of P Embodiment 24 where a secondary light source is employed containing red, green, or blue light for the purpose of photobiomodulation.

1. An endoscopic device configured to provide three-dimensional visualization of an intraocular object, the device comprising:
a first portion comprising:
a distal shaft having a lumen extending along a longitudinal axis, wherein the distal shaft has an outer diameter sized for positioning in an intraocular space;
an illumination guide extending through the lumen of the distal shaft;
at least one image transmitter extending through the lumen of the distal shaft; and
at least one objective lens positioned within the lumen at a distal end of the at least one image transmitter; and
a second portion coupled to the first portion, the second portion comprising:
a housing;
an illumination source positioned within the housing in optical communication with a proximal end of the illumination guide; and
at least one image sensor positioned within the housing in optical communication with a proximal end of the at least one image transmitter.

2. The device of claim 1, wherein the at least one objective lens is a single objective lens having an optical axis, the single objective lens configured to spatially translate relative to the distal shaft between at least a first location and at least a second location horizontally off-set from the first location, wherein the device is configured to capture images of the intraocular object as the single objective lens spatially translates to provide the three dimensional visualization.

3. The device of claim 2, wherein a distance between an optical axis of the single objective lens when positioned at the first location to the optical axis of the single objective lens when positioned at the second location is proportional to a working distance of the endoscope such that the distance defines a parallax angle for a distance from the first and second locations to the intraocular object.

4. The device of claim 3, wherein a subtended parallax angle forms two images of the intraocular object at different angular perspectives.

5. The device of claim 2, wherein the spatial translation of the single objective lens occurs side-to-side from the at least a first location to the at least a second location.

6. The device of claim 2, wherein the spatial translation of the single objective lens is rotational around the longitudinal axis from the at least a first location to the at least a second location, wherein the single objective lens is offset from the center of rotation.

7. The device of claim 2, further comprising an actuator configured to cause spatial translation of the single objective lens, the actuator being a piezoagitator or a motor.

8.-12. (canceled)

13. The device of claim 1, wherein the illumination guide comprises a first illumination guide and a second illumination guide, wherein the at least one objective lens is aligned with the longitudinal axis of the distal shaft, the first illumination guide is spatially off-set to a first side of the at least one objective lens and the second illumination guide is spatially off-set to a second side of the at least one objective lens.

14. The device of claim 13, wherein the device is configured to capture images of the intraocular object as the first illumination guide illuminates a first spatial location of the object, the second illumination guide illuminates a second spatial location of the object, and the first and second illumination guides simultaneously illuminate the object at the first spatial location and the second spatial location.

15. The device of claim 1, wherein the at least one objective lens comprises a first objective lens and a second objective lens horizontally offset from the first lens.

16. The device of claim 15, wherein a distance between an optical axis of the first objective lens and an optical axis of the second objective lens defines a parallax angle for a distance from the first and second objective lenses to the intraocular object.

17. The device of claim 15, wherein a subtended parallax angle forms two images of the intraocular object at different angular perspectives.

18. The device of claim 15, wherein the first objective lens is coupled to a first bundle of the at least one image transmitter and the second objective lens is coupled to a second bundle of the at least one image transmitter.

19.-20. (canceled)

21. The device of claim 18, wherein the distal shaft is configured to slide from a first position in which the first and second objective lenses are sheathed by the distal shaft to a second position in which the first and second objective lenses are unsheathed from the distal shaft.

22. The device of claim 21, wherein, when the distal shaft is in the second position, the first objective lens and the second objective lens move outward away from one another increasing the distance between their respective optical axes.

23. The device of claim 22, further comprising a shape memory element configured to urge the first and second objective lenses to move outward from one another upon withdrawing the distal shaft into the second position.

24.-25. (canceled)

26. The device of claim 23, wherein a maximum outer dimension of the first and second objective lenses in the second position is greater than an inner diameter of the distal shaft.

27.-38. (canceled)

39. The device of claim 1, wherein the at least one objective lens is a ball lens or a liquid lens.

40. The device of claim 1, wherein the outer diameter of the distal shaft is 0.020 inches to 0.072 inches.

41.-47. (canceled)
